(19) United States
(12) Patent Application Publication
(10) Pub. No.: US 2019/0038384 A1
(43) Pub. Date: Feb. 7, 2019

(54) METHOD AND SYSTEM FOR MAKING AND USING AN OCCLUSIVE BARRIER FOR BONE REGENERATION AND OCCLUSIVE BARRIER OBTAINED BY SAID METHOD

(71) Applicants: Mauricio Alberto LIZARAZO ROZO, Austin, TX (US); Kevin Irons, Austin, TX (US); David Yu, Austin, TX (US); Juan Vesga, Austin, TX (US)

(72) Inventors: Mauricio Alberto LIZARAZO ROZO, Austin, TX (US); Kevin Irons, Austin, TX (US); David Yu, Austin, TX (US); Juan Vesga, Austin, TX (US)

(21) Appl. No.: 16/028,663

(22) Filed: Jul. 6, 2018

Related U.S. Application Data
(63) Continuation-in-part of application No. 15/511,218, filed on Mar. 14, 2017.

(51) Int. Cl. A61C 8/02; A61C 17/00; A61C 13/00; A61B 6/14

(52) U.S. Cl. CPC A61C 8/0006; A61C 17/00; A61C 13/0007; A61C 13/0018; A61B 6/032; A61C 13/0004; A61C 13/0012; A61B 6/14; A61C 13/0019

(57) ABSTRACT

A method and system for bone tissue regeneration in association with a predetermined dental bone structure obtains a computerized tomography scan of a dental bone structure on which to regenerate bone tissue. A three-dimensional model digitally represents the dental bone structure. A treatment plan corresponding to said three-dimensional model and a design order permit forming an occlusive barrier for covering the portion of the dental bone structure whereupon to regenerate bone tissue. An occlusive barrier from a biocompatible material and an osteoconductive material form flesh and regenerated bone tissue via osteoconduction. The occlusive barrier includes irrigation channels for permitting flushing of said interior volume and may be formed of pieces printed for forming a volume to regenerate bone tissue. The occlusive barrier may be subjected to a heat treatment for alleviating molecular stress and increasing occlusive barrier ductility and strength. Surface sandblasting treatment forms surface porosity promoting osteoconduction.

### CROSS REFERENCE TO RELATED APPLICATION

This application claims the benefit of the following non-provisional application, all of which is here expressly incorporated by reference in their entirety:

Ser. No. 15/511,218 entitled “METHOD FOR PRODUCING AN OCCLUSIVE BARRIER FOR BONE REGENERATION AND AN OCCLUSIVE BARRIER OBTAINED BY MEANS OF SAID METHOD,” filed on Mar. 14, 2017 with Attorney Docket No. IRON001USN;

PCT/ES2016/070289 entitled “Meted de Fabricacion de Una Barrera Oclusiva Para Regeneracion Osea y la Barrera Oclusiva Obtenida Mediante Dicho Metodo,” filed Apr. 21, 2016, claiming priority to Spanish application serial no. P201500309, filed on Apr. 23, 2015.

### FIELD OF THE PRESENT DISCLOSURE

The present disclosure relates to methods and systems producing an occlusive barrier for bone regeneration and an occlusive barrier obtained by means of said method. More particularly, the present disclosure includes an occlusive barrier in the form of a biomedical device custom made for the patient, designed by computer and manufactured by titanium laser sintering technology, and which adapts to the measurements of the anatomical structure of the patient. The presently disclosed subject matter offers an occlusive barrier for creating a space between bone tissue and gingival tissue to promote bone growth from the layer of stem cells covering the outer surface of the bone (endosteum), which stem cells may form the basis for further tissue formation and bone regeneration.

### BACKGROUND OF THE PRESENT DISCLOSURE

At present, and for several years now, the treatment of choice to regenerate alveolar bone necessary for the placement of dental implants is called Guided Bone Regeneration (GBR). Guided Bone Regeneration (GBR) can also be defined as a bone regeneration technique by inhibition of soft tissue proliferation, by exclusion with a barrier membrane, after filling the defect with bone grafts or other filling material in order to prevent soft tissue collapse.

Grafts and bone fillings are purported to have a mechanical and biological function. In the host to bone graft interface, there is a complex relationship where multiple factors can intervene for either a successful or for unsuccessful non-incorporation of the graft. Among them, graft vascularization, local factors, systemic factors and biologic compatibility properties (depending on the type, size and shape of the graft used). Adequate bone volume for osseointegration is essential for implant therapy. One of the critical components of the stomatognathic system is the alveolar bone, which is an odonto-dependent structure, since it forms along with the dental elements and holds the teeth while fulfilling their function and resorbs away once the teeth are lost.

Among the materials used for bone regeneration are described those for filling or grafting (biological products that fill the bone defects); and among these materials autologous grafts, allogeneic materials, xenogeneic, bone substitutes, guided bone regeneration techniques and the use of bone morphogenetic proteins are included.

In this sense, the various materials used can work with at least one of known mechanisms or processes:

(a) Osteogenesis: Synthesis of new bone from cells derived from the graft or host. Requires cells capable of generating bone.

(b) Osteoinduction: The process by which osteogenesis is induced and regularly seen in any type of bone healing process. Osteoinduction implies the recruitment of immature cells and the stimulation of these cells to develop into preosteoblasts. In a bone healing situation such as a fracture, the majority of bone healing is dependent on osteoinduction.

(c) Osteoconduction: Osteoconduction means that bone grows on a surface of the graft material. This phenomenon is regularly seen in the case of bone implants. Implant materials of low biocompatibility such as copper, silver and bone cement shows little or no osteoconduction.

(d) Osseointegration is the stable anchorage of an implant achieved by direct bone-to-implant contact. In craniofacial implantology, this mode of anchorage is the only one for which high success rates have been reported. Osseointegration is possible in other parts of the body, but its importance for the anchorage of major arthroplasties is under debate. Ingrowth of bone in a porous-coated prosthesis may or may not represent osseointegration.

It is a process by which the graft material provides a suitable environment, structure or physical material suitable for the apposition of new bone by a predictable pattern, determined by the graft biology and the mechanical environment of the graft-host interface.

Ideal bone grafts and fillings should have properties of these three processes, in addition to being biocompatible and providing mechanical stability. Biocompatibility can be defined when a material is considered compatible and only causes desired or tolerable reactions in the living organism.

In order to achieve some of the processes named above, bone grafts have been studied for more than four decades. Among the different options are autologous or autogenous grafts. With autogenous grafts, bone obtained from the patient and for this reason there is little antigenic capacity. It is obtained from intraoral sites (chin, maxillary tuberosity, ascending branch) that are used for small defects or extra-oral (iliac crest, rib tibia or calvaria) when more is required. The choice of each approach will depend on the type, size and shape of the bone cavity, clinical experience and professional preference.

Allogeneic grafts or allografts: These are from individuals of the same species, but genetically different They may be classified according to their processing as:

(i) Frozen allografts.

(ii) Lyophilized allograft (freeze-dried).

(iii) Freeze-dried and demineralized allografts.

(iv) Irradiated bone.

The advantages of the allograft include its availability in significant amounts, in different shapes and sizes, no sacrificing of host structures and no donor site morbidity. Disadvantages are related to the quality of the regenerated bone tissue, which is not always predictable. A process to eliminate their antigenic capacity is needed.

Heterologous grafts or xenografts: These are of natural origin, from another species (animal) and contain the natural minerals of the bone. For example, bovine bone and coral derivatives (Nu-Oss, Osteogen, Bio-Oss, Interpore).

Alloplastic or synthetic grafts: These are synthetically manufactured materials. They are found in various shapes, sizes and textures. Biological bone responses will depend on the manufacturing techniques, crystallinity, porosity and degree of resorption.

They may include ceramic, which are the most commonly used, for example synthetic calcium phosphate (hydroxyapatite and tricalcium phosphate). Polymers, such as Bioplan, HTR may be used. Bioactive ceramic glass, composed of calcium and phosphate salts, and sodium and silicon salts (Bioglass, Perioglas, Biogran) may also have use.

All implantation material should trigger a reaction that is as physiologically compatible with the surrounding tissues. It is essential to know the normal biological processes that are triggered in the regeneration and the physical, mechanical and biological characteristics of each material.

At present, to use autologous grafts, a surgical procedure is required at the donor site for its production, with the consequent risk of postoperative morbidity, infection, pain, hemorrhage, muscle weakness, neurological injury, graft necrosis, among others; in addition, the surgical time is considerably increased and in some cases the amount of graft generated may be insufficient.

In the current technique of bone regeneration, the professional must preform by hand the device that he will implant in the patient. When the devices or barriers are preformed, spaces may remain that allow soft tissues to be invaded as well as the entrance of bacteria, with the consequent risk of infection and therefore of treatment failure. This process is seriously lacking in efficiency and accuracy.

At present, in guided bone regeneration (GBR), the time required for the reabsorption of the bone material used for grafting or filling determines the formation of the new tissue. For example, in the case of Cerasorb® tricalcium phosphate (synthetic ceramic graft) the average time for resorption is 24 to 36 months; and in the case of Bio-Oss® (heterologous graft of bovine bone), because it is a ceramic material, it is not absorbed but over time because it forms a mixture between the filling material and bone. In order for implants to be placed, turnover should be less than months for osseointegration, and thus proceed with the patient's dental rehabilitation. GBR processes which use flexible membranes collapse under their own weight is responsible for a decrease in the volume of bone required in the regeneration.

### BRIEF SUMMARY OF THE PRESENT DISCLOSURE

The present disclosure produces an occlusive barrier for bone regeneration and an occlusive barrier obtained by means of the disclosed method. More particularly, the present disclosure includes an occlusive barrier in the form of a biomedical device custom made for the patient, designed by computer and manufactured by titanium laser sintering technology, and which adapts to the measurements of the anatomical structure of the patient. Occlusive barriers belong to the sector that involves additive techniques such as laser sintering and subtractive ones such as computerized machining, as applied in medical sciences such as dentistry.

In one aspect of the disclosure, a method, system, and integrated medical system is disclosed for bone tissue regeneration in association with a predetermined dental bone structure. The disclosure includes the steps of obtaining a computerized tomography scan of a dental bone structure on which to regenerate bone tissue. A three-dimensional model formed from the computerized tomography scan digitally represents the dental bone structure. The method and system present the three-dimensional model on a computer display. A treatment plan corresponds to the three-dimensional model. The method and system receive a design order relating to the treatment plan for forming an occlusive barrier for covering the portion of the dental bone structure whereupon to regenerate bone tissue. An occlusive barrier is formed from a biocompatible material in accordance with said design order. An osteoconductive material is placed within an interior volume of the occlusive barrier for associating with and from which may form fresh and regenerated bone tissue via osteoconduction in association with the portion of the dental bone structure covered by said occlusive barrier. The method and system further requires fixating the occlusive barrier and the osteoconductive material to the dental bone structure for a time period sufficient for regeneration of bone tissue associated with dental bone structure. The occlusive barrier is removed from the dental bone structure upon said bone tissue regeneration reaching a predetermined stage.

The disclosed subject matter further includes an occlusive barrier for osteoconductive bone tissue regeneration in association with a predetermined dental bone structure. The occlusive barrier may be formed by performing the steps of obtaining a computerized tomography scan of a dental bone structure on which to regenerate bone tissue. A three-dimensional model from said computerized tomography scan for digitally represents the dental bone structure. The occlusive barrier may include a plurality of irrigation channels for permitting flushing of said interior volume during regeneration of bone tissue associated with dental bone structure.

The occlusive barrier may be specified using a computer-aided design application and manufactured by a titanium laser sintering process. The occlusive barrier further includes a space between the predetermined dental bone structure tissue and gingival tissue for promoting bone growth from a layer of stem cells covering an outer endosteum outer surface of the dental bone structure. The occlusive barrier may be formed from a plurality of pieces printed for associating into said occlusive barrier covering said portion of the dental bone structure whereupon to regenerate bone tissue. The occlusive barrier is subjected to a heat treatment for alleviating molecular stress and increasing occlusive barrier ductility and strength and may be subjected to a surface sandblasting treatment for forming a surface porosity promoting osteoconduction and blood vessel formation. Furthermore, the occlusive barrier is subjected to an anodizing treatment for cleaning organic and inorganic residues from surfaces of said occlusive barrier.

A technical advantage of the present disclosure includes the ability to obtain a computerized tomography scan of a dental bone structure on which to regenerate bone tissue through an internet web application.

Another technical advantage of the present disclosure includes the ability to form a three-dimensional model from said computerized tomography scan for digitally representing the dental bone structure using a three-dimensional printing software application.

Still a further technical advantage of the present disclosure includes the ability to present the three-dimensional model on a computer display for viewing said three-dimensional model from a multitude of three-dimensional perspectives.

A technical advantage of the present disclosure includes the ability to receive a treatment plan corresponding to said three-dimensional model from a remote location corresponding to a certified dental surgeon office at a remote location through a web portal or smart device

The disclosed subject matter further includes receiving a design order relating to said treatment plan for forming an occlusive barrier for covering the portion of the dental bone structure whereupon to regenerate bone tissue, said design order specifically relating to an individual patient for whom said occlusive barrier may be custom-fabricated.

### BRIEF DESCRIPTION OF THE DRAWINGS

The present subject matter will now be described in detail with reference to the drawings, which are provided as illustrative examples of the subject matter so as to enable those skilled in the art to practice the subject matter. Notably, the figures and examples are not meant to limit the scope of the present subject matter to a single embodiment, but other embodiments are possible by way of interchange of some or all of the described or illustrated elements and, further, wherein:

FIG. 1 shows an occlusive barrier that is an object of the present disclosure according to a preferred embodiment;

FIG. 2 shows an occlusive barrier that is an object of the present disclosure according to another preferred embodiment;

FIG. 3 shows another view of the occlusive barrier shown in FIG. 1;

FIG. 4 shows a sectional view of the occlusive barrier according to the preferred embodiment shown in FIGS. 1 and 3;

FIGS. 5A through 5C, 6 and 7 show the occlusive barrier that is the object of the present disclosure according to yet another preferred embodiment;

FIGS. 8A through 8F illustrate additional aspects of the illustrated embodiments of the present disclosure;

FIG. 9 shows an occlusive barrier process according to one aspect of the present disclosure;

FIG. 10 illustrates a patient's mouth wherein the methods, system and structure of the present disclosure may have application;

FIG. 11 illustrates a frontal and side X-ray view of the patient's jaw for the example of FIG. 10;

FIG. 12 shows a frontal and side x-ray of a patient frontal jaw bone structure prior to the frontal jaw bone structure being prepared for the procedure of the present disclosure;

FIG. 13 depicts a computerized model of a patient frontal jaw bone structure from FIG. 9, wherein the CT scan has been converted to a three-dimensional computer aided design image;

FIG. 14 provides a photograph of an exemplary occlusive barrier for use with the patient of FIG. 9;

FIG. 15 illustrates the preparation of the patient frontal jaw bone structure for use of the occlusive barrier consistent with the method and system of the present disclosure;

FIG. 16 illustrates the extraction of teeth to provide a sufficient region for use of the occlusive barrier of the present disclosure;

FIG. 17 shows the interior portion of the occlusive barrier of FIG. 14 and how the occlusive barrier may include a congealed blood clot for placement within the occlusive barrier;

FIG. 18 illustrates the placement of the occlusive barrier covering the lower teeth of the front of the patient's jaw;

FIG. 19 further illustrates the procedure whereby tissue removed for opening the area for placement of the occlusive barrier is being restored and used to cover the occlusive barrier;

FIG. 20 illustrates a surgical suture to provide for tissue regeneration and growth with the use of the occlusive barrier;

FIG. 21 illustrates a removable provisional prosthesis (Essix retainer) over the region of the jaw including the occlusive barrier during the healing process, the dental covering will allow the patient to use his teeth in a relatively unrestricted way;

FIG. 22 illustrates the bone regeneration made possible by the procedure of the present disclosure;

FIG. 23 illustrates a user interface for the occlusive barrier information management and design, and structural design software for use with the process of the present disclosure;

FIG. 24 depicts case management features of the method and system of the present disclosure for providing a patient-oriented service with the occlusive barrier construction;

FIG. 25 depicts a CT scan based three-dimensional model of a frontal jaw bone structure as provided by the software method and system of the present disclosure for use by a dentist in managing the design of an occlusive barrier;

FIG. 26 illustrates various tools and aids in the design of the occlusive barrier for the patient's jaw;

FIG. 27 provides additional illustrative tools and ways of using the patient's CT scan-based three-dimensional image for permitting a skilled dental surgeon to prepare an occlusive barrier for performing the method and system of the present disclosure;

FIG. 28 depicts how a user (i.e., a skilled dental surgeon, or dentist) may use the software of the present disclosure for identifying and specifying an occlusive barrier has herein disclosed;

FIG. 29 illustrates the results that the method and system of the present disclosure provide with an occlusive barrier design and construction algorithm;

FIG. 30 illustrates further aspects of the occlusive barrier and the design for construction the titanium portion of the barrier;

FIG. 31 illustrates aspects of using insert or set screws to place an occlusive barrier on a patient's jaw in the formation of the structure for achieving the objectives and features of the present disclosure;

FIGS. 32 and 33 illustrate the three-dimensional image manipulation aspects of the software presented as part of the disclosed method and system for permitting the dental practitioner to see and manage the construction of the occlusive barrier according to a patient's needs; and

FIGS. 34 through 37 provide a further information on automation aspect of the present disclosure whereby a website is made available to patients or professionals in designing the occlusive barrier and using the software for the present method and system.

### DETAILED DESCRIPTION OF ILLUSTRATIVE EMBODIMENTS

The detailed description set forth below in connection with the appended drawings is intended as a description of exemplary embodiments in which the presently disclosed process can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed method and system. However, it will be apparent to those skilled in the art that the presently disclosed process may be practiced without these specific details. In some instances, well-known structures and devices are shown in block diagram form in order to avoid obscuring the concepts of the presently disclosed method and system.

In the present specification, an embodiment showing a singular component should not be considered limiting. Rather, the subject matter preferably encompasses other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present subject matter encompasses present and future known equivalents to the known components referred to herein by way of illustration.

The occlusive barrier is a biomedical device custom made for the patient, designed by computer and manufactured by titanium laser sintering technology, which adapts to the measurements of the anatomical structure of the patient. The object of the occlusive barrier is to create a space between the bone tissue and the gingival tissue to promote bone growth from the layer of stem cells that is covering the outer surface of the bone (endosteum). Its function, therefore, is to maintain the space to support the clot, which ultimately achieves tissue regeneration. This structure or biomedical device makes it possible to maintain stability of the clot and isolated it from the external environment, also avoiding bacterial invasion that would impede the regeneration process.

Accordingly, the occlusive barrier is printed on one or more pieces. With the occlusive barrier being formed of more than one piece, the occlusive barrier comprises a concavity in at least two of the pieces, each of the recesses being configured to partially enclose a tooth and so form the concavities that together surround the tooth. In this way, said pieces are complementary to jointly define at least one through hole into which the tooth may be fitted.

Biocompatibility is defined as the ability of a material to act with a suitable response to the host, in a specific application. This type of material is known as a biomaterial used for the service of medicine, in this case dentistry, to interact with biological systems inducing a specific biological activity.

The reasons for considering titanium as the ideal biomaterial in the making of custom occlusive barriers are numerous. They include the fact that titanium is inert. The oxide covering in contact with the tissues is insoluble, so that no ions that might react with organic molecules are released. Also, titanium in living tissues acts as a surface upon which bone may grow and adhere to the metal.

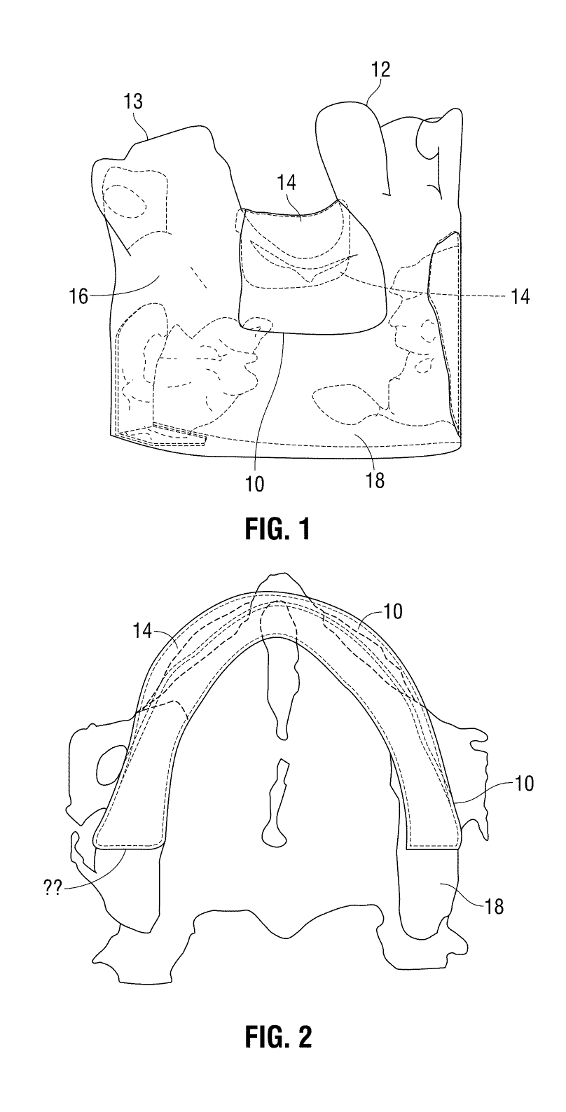

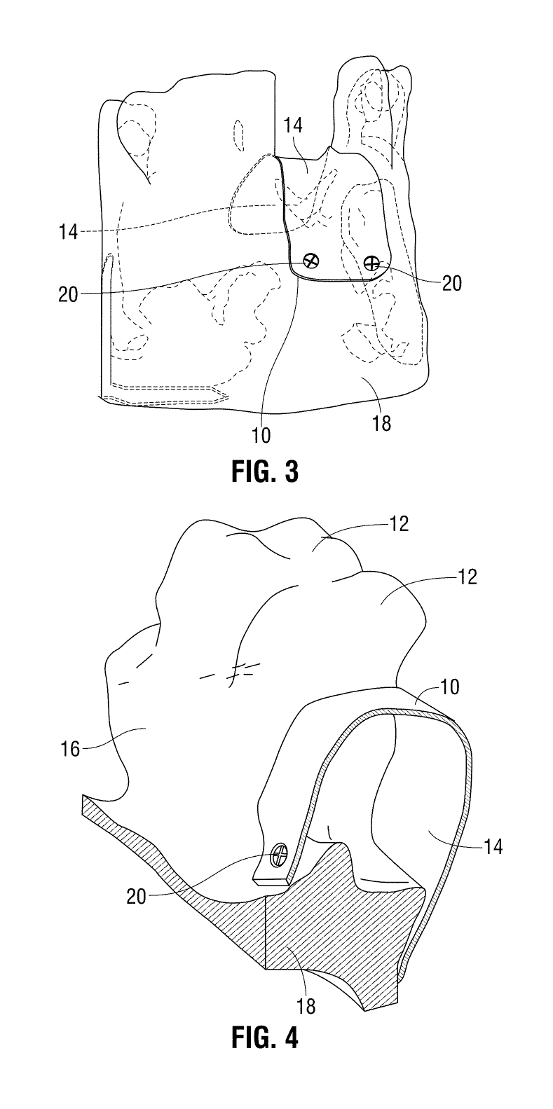

FIG. 1 shows occlusive barrier 10 positioned between teeth 12 and 13 at regeneration region 14. Occlusive barrier 10 attaches to gingival tissue 16 to provide a barrier region for bone regeneration at bone regeneration region 14, Which is a segment of jaw bone tissue 18. FIG. 2 provides another perspective of an occlusive barrier 10 consistent with the teachings of the present disclosure, where occlusive barrier 10 may cover a significant portion of frontal jaw bone tissue 18 to provide regenerative bone growth in a bone regeneration area 14. FIG. 3 provides a further illustration of how jaw bone regeneration region 14. Occlusive barrier 10 may be positioned using set screws 20.

In FIGS. 1 through 3, occlusive barrier 10 is individualized for each patient. Occlusive barrier 10 makes reconstruction of new bone tissue possible and/or is used to replace and regenerate destroyed or lost structures, and without the need to use filler materials or bone grafts. Occlusive barrier 10 works as a biomedical device and induces new bone formation by acting as a biological barrier to prevent migration of epithelial cells from connective tissue and bacteria that would cause inhibition of bone growth. Occlusive barrier 10 is fully adapted to the surgical site, having the capacity to maintain a total regenerative space and the possibility of vascularization. Occlusive barrier 10 ensures three-dimensional reconstruction of defects of the alveolar bone and facilitates restoration of alveolar bone by appropriate fixation of the restitution material. Occlusive barrier 10 promotes adequate space for the formation of a natural fibrin molding, a precursor of bone tissue, as may be seen in FIGS. 1 and 3.

Occlusive barrier 10 possesses the osteoconduction mechanism, since it provides an environment, structure and physical material that triggers a three-dimensional growth of capillaries, perivascular tissue and most important, the recruitment of mesenchymal stem cells in the area, for its subsequent differentiation into osteoblasts modulated by growth factors. This scaffolding allows the formation of new bone through a predictable pattern, determined by the biology and dimensions of thickness and height previously given in the design and approved by the professional.

The thickness of occlusive barrier 10 ranges between 0.3 and 0.6 millimeters. Lower values hinder the ability to maintain the space, but higher values make it difficult for the patient to accept the occlusive barrier in the corresponding fixation area.

FIG. 4 shows a sectional view of the occlusive barrier 10 according to the preferred embodiment shown in FIGS. 1 and 3. Set screws 20 also appear in FIG. 4 where bone regeneration region 14 is covered by occlusive barrier 10 adjacent teeth 12 and over gingival tissue 16.

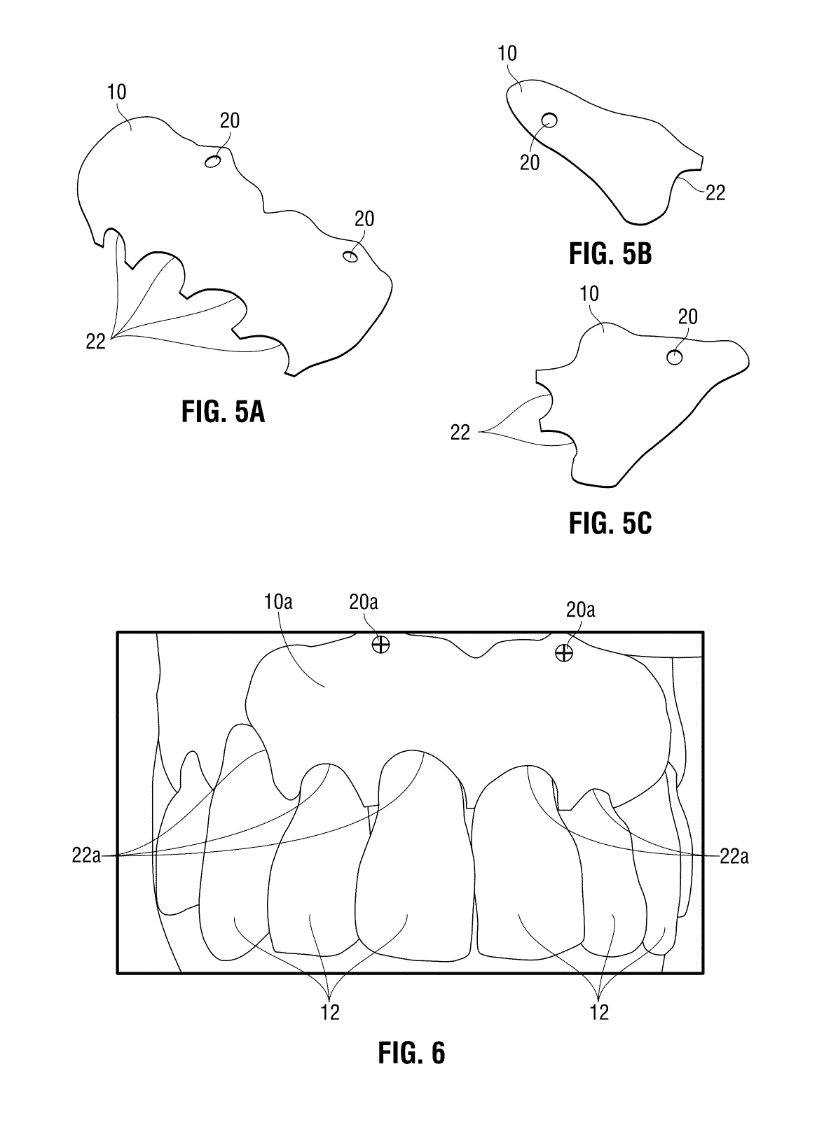

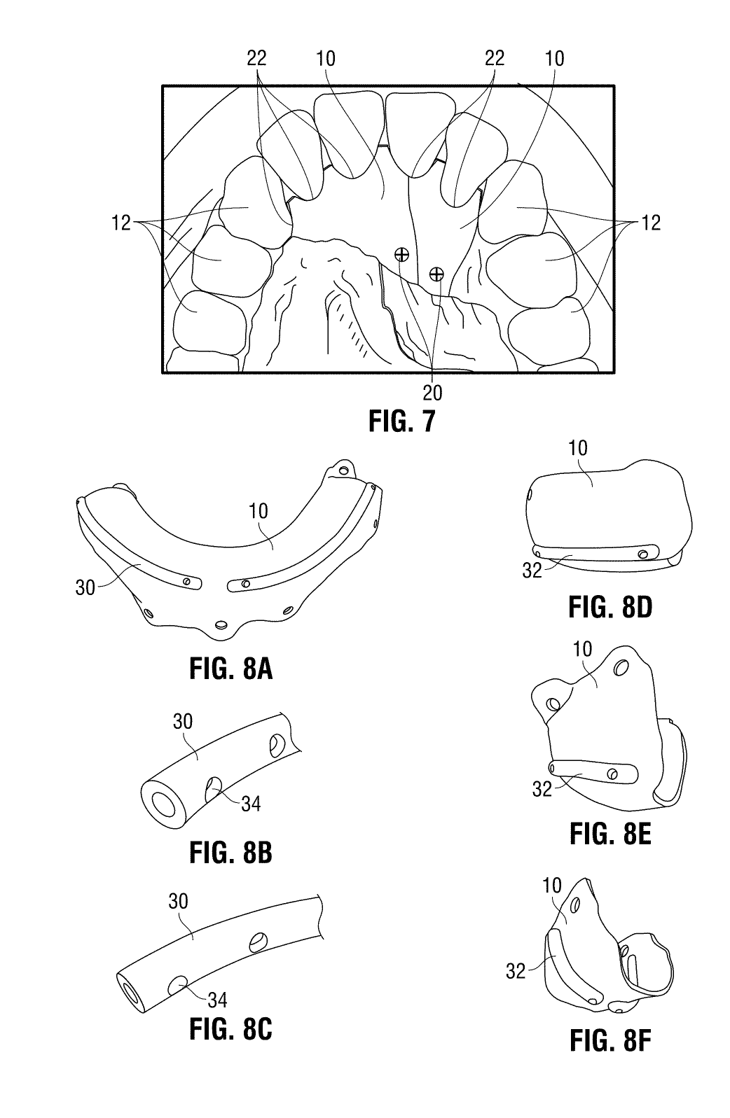

FIGS. 5A through 5c, 6 and 7 show the occlusive barrier that is the object of the present disclosure according to yet another preferred embodiment. FIGS. 5A-5C, thus, illustrate the characteristic that occlusive barrier 10 may include numerous segments. For example, segment A may be designed to cover the front part of a frontal jaw region above a patient's teeth, as shown in FIG. 6. In addition, parts B and C maybe further configured to provide the necessary occlusive barrier 10 to support bone regeneration on the interior side of the jaw bone behind part A. Occlusive barrier 10 of FIGS. 5A-5C and 6 show, in particular, the dental barriers 22 that may be formed around the patient's teeth as part of the design that occlusive barrier 10 enables. FIGS. 5A-5C and 6 further show set screws 20 that may be used to fasten occlusive barrier 10 to the patient's jaw during the bone regeneration process. FIG. 7 continues the illustration of FIGS. 5A-5C and 6 to show that occlusive barrier 10 parts B and C may be fastened behind teeth 12.

The presently disclosed occlusive barrier formation and use system includes the design and formation of the implantable occlusive barrier 10 and a jaw model used to demonstrate the system and facilitate ordering dentist or physician proper fit of occlusive barrier 10 to the patient specific jaw anatomy. However, the jaw CT scan-based model does not come in patient contact at any point. Occlusive barrier 10 is to be implanted in dental/oral tissues and removed in four to eight months after appropriate bone healing per dentist/physician's professional diagnosis. Occlusive barrier 10 is a single use product and provided non-sterile to the dentist or physician, to be steam sterilized on-site per a physician's office procedures for steam sterilization prior to implant.

The orifices, which may be seen in FIGS. 4 and 5A-5C, are configured for the insertion of screws 20, preferably of titanium. The orifices are for securing the occlusive barrier to the patient's alveolar bone. For this reason, said orifices are located in the occlusive barrier depending on the patient, and more specifically on the available bone of the patient.

Occlusive barrier 10 may be partial or total. These are defined as a function of their longitudinal extension upon the alveolar bone. Examples of the occluding barriers may be seen in FIG. 1, and FIGS. 3 to 7 when they are partial and in FIG. 2 an example is shown of the occlusive barrier when it is total. In this way, the partial occlusive barriers may be limited by the teeth at least at one of their longitudinal ends, while the total occlusive barriers cover the entire longitudinal extension of the alveolar bone in which the teeth are located and upon which they are available. The longitudinal extent of the occlusive barriers on the alveolar bone may be greater than in the case of flexible membranes because these do not collapse under their own weight as occurs with such membranes.

Occlusive barrier can be printed in one piece, as is seen in FIGS. 1 to 3, or on several pieces, as is seen in FIGS. 5A to 7. The parts forming an example of the occlusive barrier, namely three, are shown in FIG. 5A-5C, while FIGS. 6 and 7 show the arrangement of said occlusive barrier together.

FIGS. 8A through 8F illustrate additional aspects of the illustrated embodiments of the present disclosure. FIGS. 8A through 8F illustrate additional aspects of occlusive barrier 10 of the disclosed embodiments. In particular, FIG. 8A shows occlusive barrier 10 similar to the embodiment appearing above in FIG. 2. Additionally, occlusive barrier 10 of FIG. 8A includes an irrigation channel 30 that may be used to provide flushing irrigation to the bone regeneration region beneath occlusive barrier 10. FIGS. 8B and 8C show how irrigation channel 30 provides a passageway for irrigation channel flow at channel 32. In addition, FIGS. 8B and 8C show irrigation paths 34 in irrigation channel 30 that permit flow of cleansing liquid during the bone regeneration process.

FIGS. 8D, 8E, and 8F show further an occlusive barrier configuration 10 similar to that disclosed above in FIGS. 5A-5C, 6 and 7. These configurations, also, provide the irrigation canals 30 made part of occlusive barrier 10. With each of the parts A of FIG. 8D, B of FIG. 8E, and C of FIG. 8F, an irrigation canal 30 may be formed and used for providing the necessary and desired irrigation of the bone regeneration region.

Occlusive barrier 10 may be printed on one piece or on at least two pieces. With the occlusive barrier formed by more than one piece, the occlusive barrier comprises a concavity in at least two of the pieces, each of these recesses being configured to partially surround one of the teeth. In this case, the recesses together define a through hole according to the outer contour of the tooth to cover the bone while taking the tooth into account. The occlusive barrier is printed on several pieces mainly because the patient, on occasion, despite suffering from the defect or bone wear, still retains the tooth or the teeth of the affected area. The present method of manufacturing makes it possible to obtain the pieces in the most optimal form, in accordance with each case.

The pieces forming the occlusive barrier are obtainable in a complementary way to cover the all or part of the alveolar bone leaving the space corresponding to the teeth located in the area intended to house the occlusive barrier free, further leaving the corresponding gap free. In the exemplary embodiment shown in FIGS. 5A to 7, the occlusive barrier is of three pieces. As can be seen in FIG. 7, the occlusive barrier may also be formed of several pieces to better adapt to the shape of the bone and facilitate their placement, that is to say that it may be formed by the laterally complementary pieces to cover the affected area according to the longitudinal extension of the bone.

The titanium used in the creation of the occlusive barrier is a material designed to interact safely and effectively with biological systems. Biomaterial-host interactions do not present any type of safety problem for the patient, i.e., it is one hundred percent compatible. The titanium used is preferably a titanium alloy called Ti64 or Ti6Al4V, having a density of 4.43 gicm3.

Beneficial properties of medical titanium are numerous. For instance, titanium is inert, the oxide covering in contact with the tissues is insoluble, so that no ions that might react with organic molecules are released. Also, titanium in living tissue acts a surface upon which the bone grows and adheres to the metal, forming an ankylotic anchor, also called osseointegration. This reaction normally only occurs in materials called bioactive and is the best base for functional dental implants.

Titanium also demonstrates good mechanical properties. Titanium tensile force is very similar to that of the stainless steel used in surgical prostheses that are load bearing. It is much stronger than dentine or any cortical bone, thus making it possible for the implants to withstand heavy loads. Moreover, the metal is soft and malleable, which helps absorb shock loads.

Titanium is a biocompatible metal (biomaterial) because the body's tissues tolerate its presence without any allergic reactions from the immune system. This biocompatibility property of titanium coupled with its mechanical qualities of hardness, lightness and strength have made a large number of medical applications possible, not only dental implants, but also hip and knee prostheses, bone screws, anti-trauma plates, components for manufacturing heart valves and pacemakers, surgical instruments, etc.

Characteristics of occlusive barrier 10 of the present disclosure are beneficial and numerous. Occlusive barrier 10 provides cellular occlusion that has the property of being isolated from the gingival tissue of the flap that opens during surgery, from the maturation of the fibrin clot in the wound space.

In addition, occlusive barrier 10 demonstrates space holding capacity and has the ability to withstand its own collapse determined by its rigidity. That is, occlusive barrier 10 possesses the physical property of being able to withstand its own collapse determined by its rigidity, guaranteeing the predetermined bone volume in the design of the biomedical device. Tissue integration is also desirable, in that the occlusive barrier should become as integrated as possible to the tissue where it is placed.

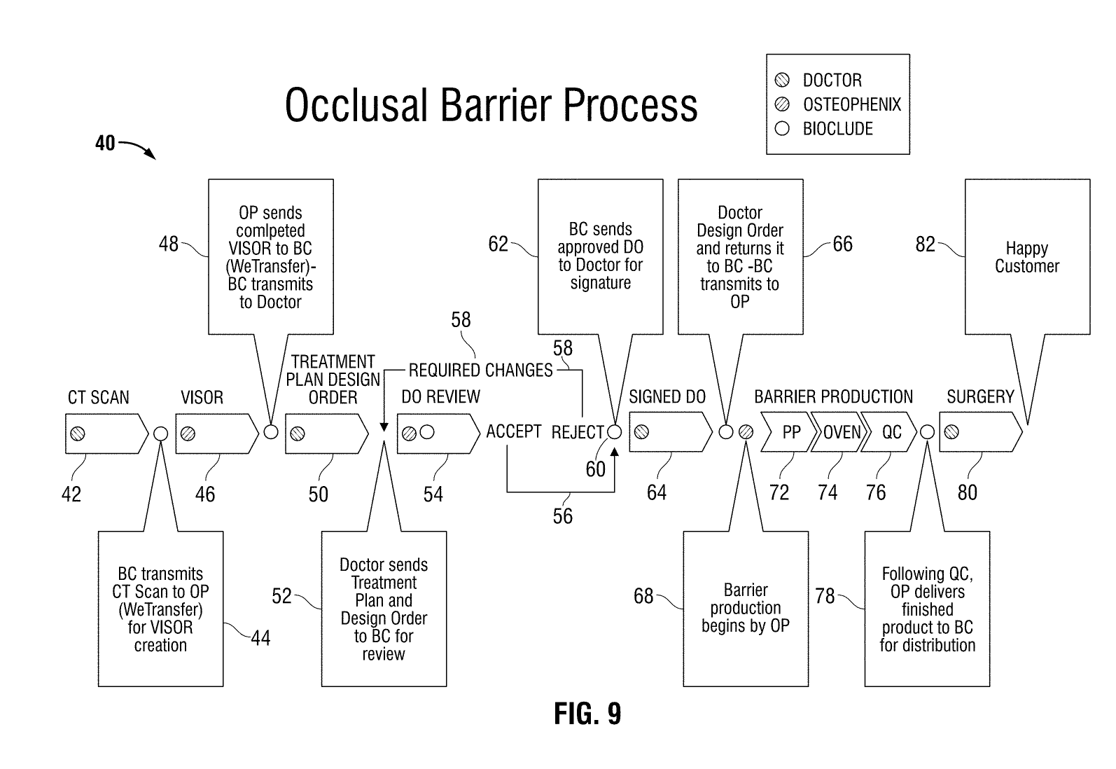

FIG. 9 shows occlusive barrier process 40 for forming and fitting occlusive barrier 10 on a patient. Beginning at step 42, occlusive barrier process 40 performs a CT scan. This would be performed at the doctor's office with the patient. The CT scan is transmitted to the occlusive barrier design center, here identified with the letters BC (for the company BiOcclude, which a company in the U.S. performing this service). The barrier design center transmits the CT scan to the barrier manufacturer, here referenced with the letters OP (for the company OstoPhoenix). This occurs at step 44.

The next step 46 includes visor creation at stage 46. The visor is formed at point 48 by the barrier manufacturer, OP, which then sends the completed visor to BC. BC then s the visor to the doctor treating the patient. Next, OP sends the completed visor to the BC, at which point the BC transmits the visor to the treating doctor. At stage 50, the treatment plan and design order are generated by the doctor, who then transmits the treatment plan and design order, at step 52, to BC for review.

At stage 54, a review occurs with both the OP and BC. Once the treatment plan and design order are accepted by OP and BC, process flow continues at 56 top point 60, whereupon BC sends the approved design order to the doctor for signature. Alternatively, if the design order is rejected by OP, required changes occur and process returns to point 52 for further review and ultimate acceptance.

At stage 64, the doctor signs the design order and then returns it to BC. Then, BC transmits the design order to OP. Then, at step 68, occlusive barrier 10 production begins by OP. At stage 70, the production of occlusive barrier 10 includes PP step 72, oven step 74, and quality control or QC step 76. Once stage 70 is completed, process flow continues to point 78, where OP manufacturers and delivers the finished product of occlusive barrier to BC for distribution. At step 80, patient surgery occurs with the doctor and the process of bone regeneration can begin.

Certain aspects of occlusive barrier process 40 are important to consider. For example, as the tomography is obtained, and it is then sent to the BC. There, noise is cleaned up and the CT scan is converted into a three-dimensional file that can be inserted into any CAD modeling software. Once this CAD file is obtained, it is imported into the modeling software, where it is located and drawn upon, to define the area of the occlusive barrier and to generate a surface that will become the occlusive barrier. Once approved and corrected by the doctors, the occlusive barrier is exported for manufacturing.

For manufacturing, the exported design file is used and is made into a post-processed file in a CAM software, which converts it into layers for titanium printing. Occlusive barrier 10 is printed in layers of 30 or 60 microns thick and, once printed, is passed to a machining stage. In this phase, occlusive barrier 10 is initially subjected to a heat treatment to alleviate the molecular stress and to make the occlusive barrier more ductile and strong, and then subjected to a surface sandblasting treatment which makes it possible for the occlusive barrier to have the optimum characteristics for osseointegration. By sandblasting the surface, a porosity is achieved to promote osteoconduction and the formation of blood vessels around occlusive barrier 10. In this way, occlusive barrier 10 with a mean arithmetic roughness (Ra) of 9-12 μm and a mean roughness range (Rz) of 40-80 μm is obtained.

Finally, the thicknesses, the orifices, and dimensions in occlusive barrier 10 are checked in a highly accurate optical gauge, to ensure that the occlusive barrier has the geometric characteristics initially defined. From there it goes to a sterilization treatment to be packed and sent to the customer.

Occlusive barrier 10 is subjected to an anodizing treatment. By means of this anodizing treatment, both organic and inorganic residues are cleaned from the surface, thus obtaining better resistance against corrosion, a decrease in the release of titanium ions to the physiological medium, greater surface hardness, improvement in the properties of osteoconduction and a coloration similar to that of the gums. Coloration is important in cases where the occlusive barrier is exposed after placement in the patient to reduce the associated visual impact.

BBS design involves acquisition of patient CT data. A dentist/physician acquires CT imaging of patient jaw/oral anatomy per their standard protocols. Then, the process includes exporting of DICOM files (0.16-0.2 mm voxel sizes). This includes transmission of patient CT data (DICOM) to the occlusive barrier designer. A dentist/physician orders an occlusive barrier and jaw model for manufacture per the patient specific CT imaging.

This includes transmission of patient CT data (DICOM) with physician directed treatment plan from the occlusive barrier designer to an occlusive barrier manufacturer who generates a three-dimensional model using already 510(k) cleared software. The occlusive barrier contract manufacturing uses already 510(k) cleared software (e.g., 3matic, k060950 branded algorithms) in conversion of patient's CT images and design of proposed barrier and jaw model.

Final authorization is by the dentist/physician to begin build the patient's occlusive barrier. The build model is communicated to occlusive barrier designer and then back to ordering dentist/physician. If the order is accepted by dentist/physician, the contract manufacturing finalizes design files for additive manufacturing and manufactures, post-processes products (occlusive barrier and jaw model). If the order is not accepted by dentist/physician, changes requested are communicated to the barrier designer and then back to the contract manufacturer. These steps may be repeated until order acceptance occurs. Once the occlusive barrier design is accepted, manufacturing occurs, as well as the assignment of individualized patient implant order marking, packaging and shipment.

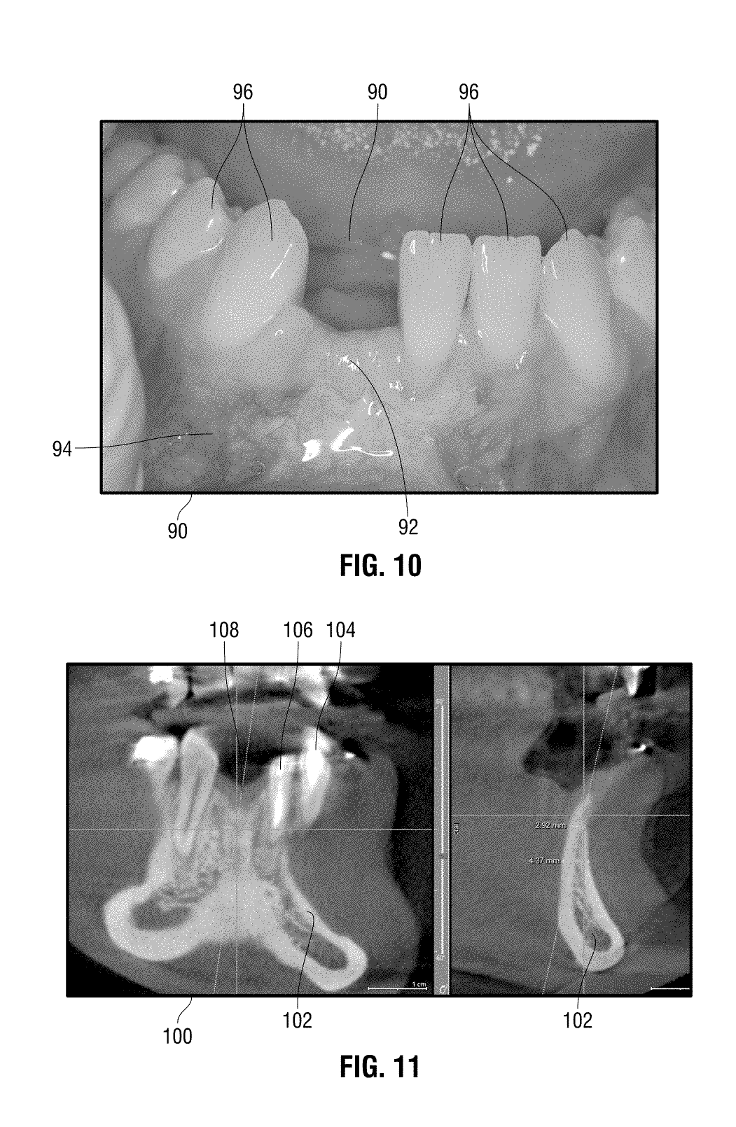

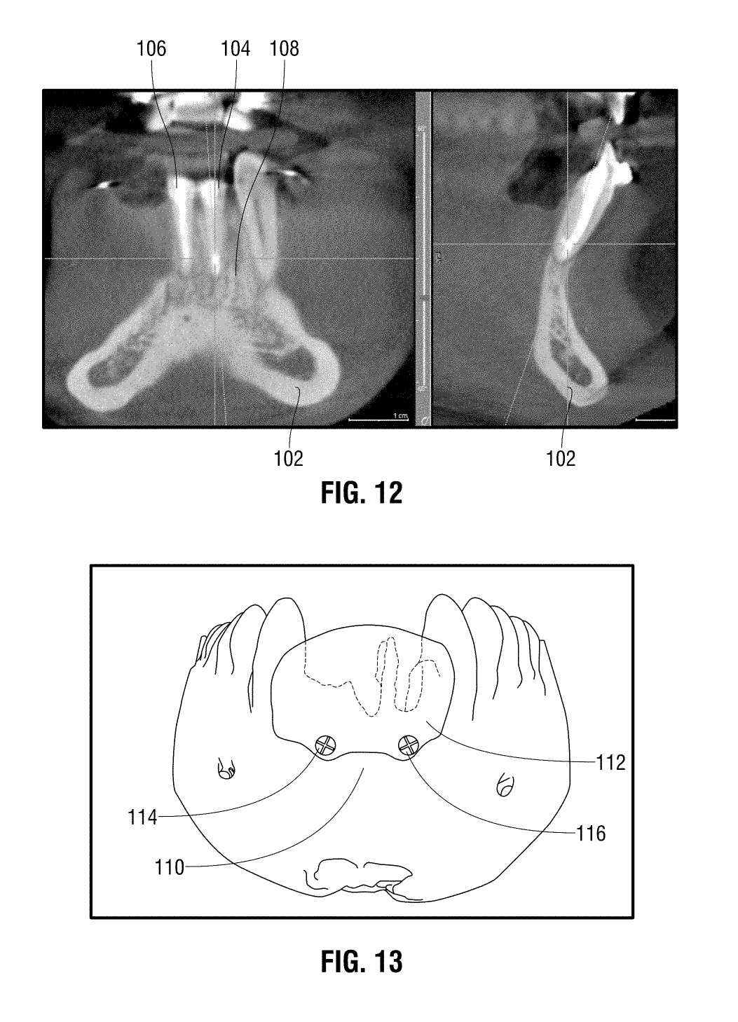

FIG. 10 shows a patient jaw 90 where there is the need for bone regeneration in regeneration region 92. Gingival tissue 94 is seen in this region as well. For the patient having teeth 96, the occlusive barrier process 40 and occlusive barrier 10 of the present disclosure will be beneficial to support jaw bone regeneration. This section is seen further in the CT scan images of FIG. 11, which show a front view CT scan and a side view CT scan. The front view CT scan shows bone regeneration region 108 where to the right appear teeth 106 and 104 which will be removed for inclusive barrier 10. FIG. 12 further shows a CT scan where front region 108 shows the interior view of jaw bone 102 with bone regeneration region 108 and teeth 104 and 106.

FIG. 13 shows a three-dimensional CT scan illustrating how the method and system from occlusive barrier replacement here describe create a digital template for the formation and guiding the medical procedure in the installation of the occlusive barrier 10. In addition, the digital image of FIG. 13 illustrates a digital template for occlusive barrier 10 in the form of image 112, as well as set screws 114 and 116.

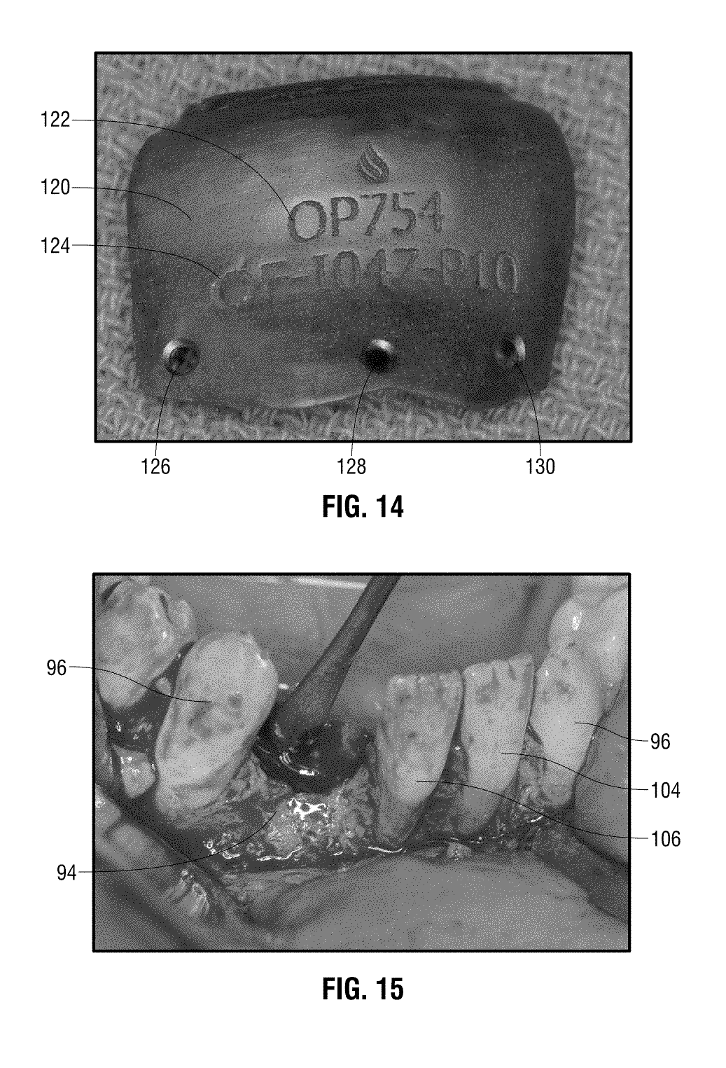

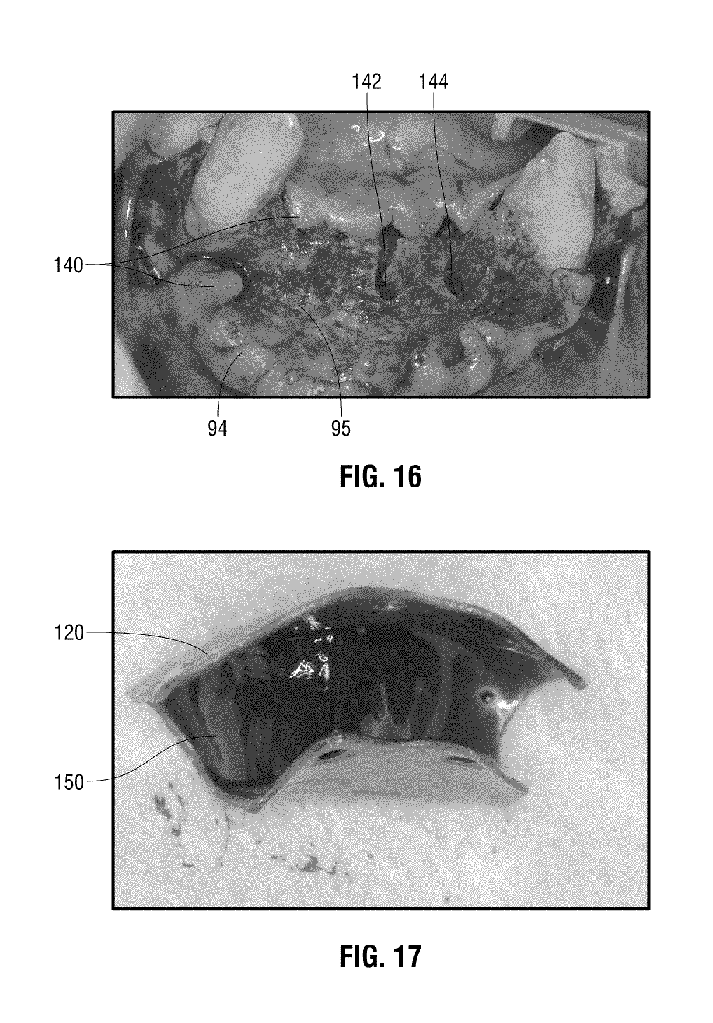

FIG. 14 shows occlusive barrier 120 formed by three-dimensional printing process, which has been specifically made to adapt to the jaw bone regeneration region of a specific patient. Occlusive barrier 120 of FIG. 14 includes specific identification reference numerals 124, as well as set screw holes 126, 128 and 130. FIG. 15 shows further preparation for the medical procedure of installing occlusive barrier 120. In preparation, gingival tissues 94 around bone regeneration region 94 is surgically separated. In addition, teeth 104 and 106 are removed. This is shown in FIG. 16. For this procedure, gingival tissue 94 is further separated at the bone regeneration region and where teeth 104 and 106 have been removed. This appears more clearly in light of the gap at 140 showing the difference between the distance between the two gingival tissue segments 94.

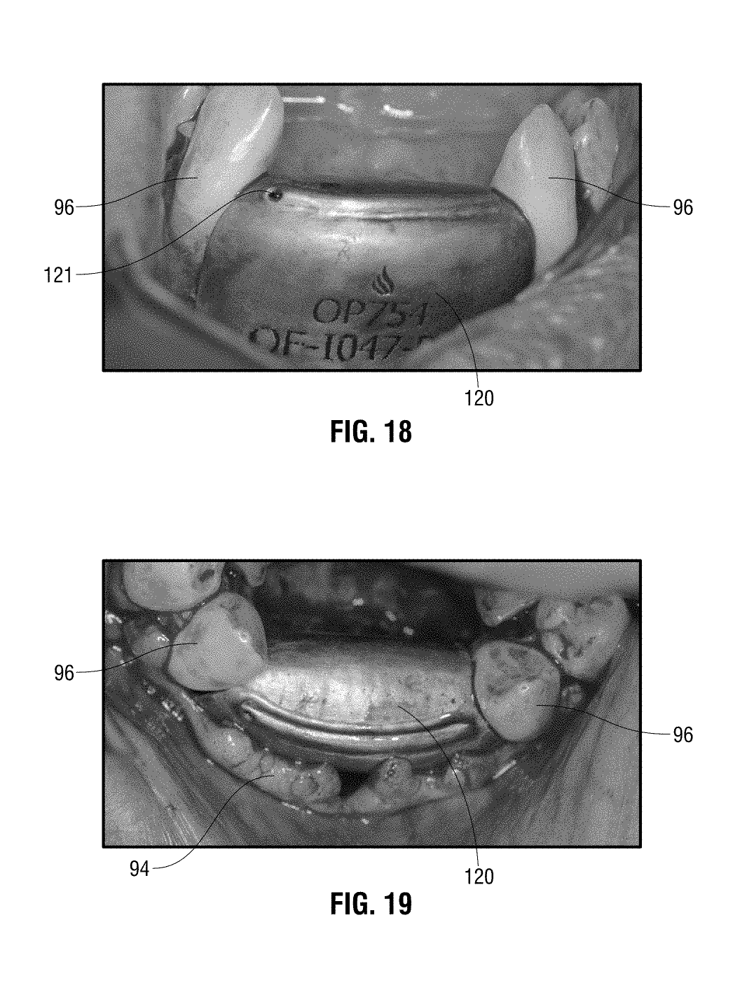

FIG. 17 shows in more particularity occlusive barrier 120 just prior to placement. Note that congealed blood clot 150 has been added to the interior portion of a close a barrier 120. FIGS. 18 through 20 show the further steps in installing occlusive barrier 120 according to the present procedure. FIG. 18 shows that occlusive barrier 120 precisely fits between teeth 96 and over the bone regeneration region for an air-tight fit. Also note irrigation canals 121 for supporting the irrigation of the bone regeneration region beneath occlusive barrier 120.

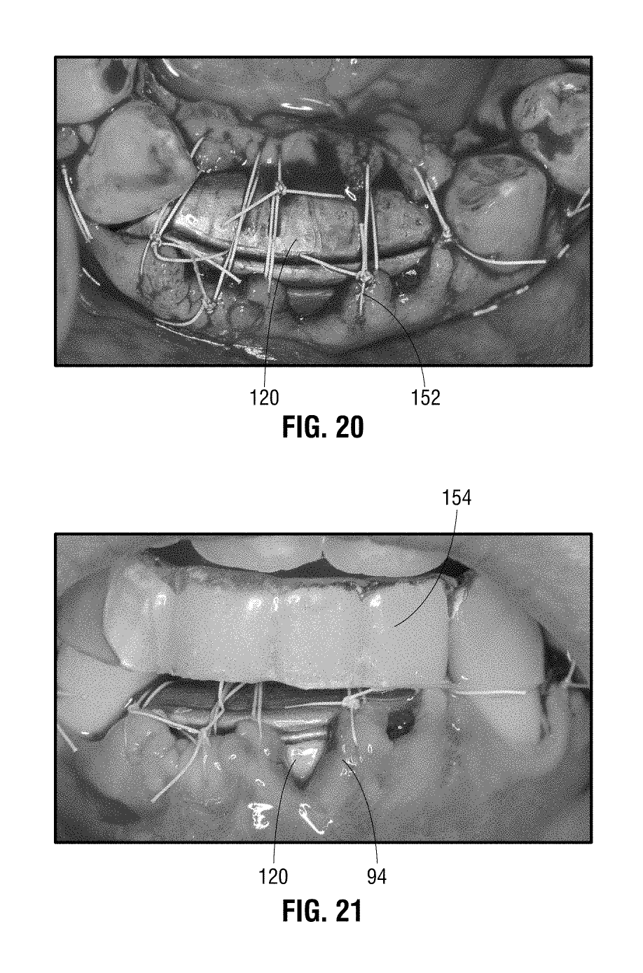

FIG. 19 further details the placement of occlusive barrier 20 showing the gingival tissue 94 being placed on both sides of occlusive barrier 120. Next, as FIG. 20 shows, surgical sutures are used to place the separated gingival tissue over occlusive barrier 120. Then, as FIG. 21 depicts a segment 154 of artificial teeth (Essix retainer) are installed over occlusive barrier 120 and the sutured gingival tissue 94. This final step places the bone regeneration configuration in the condition so the patient's body can heal perform a bone regeneration stage.

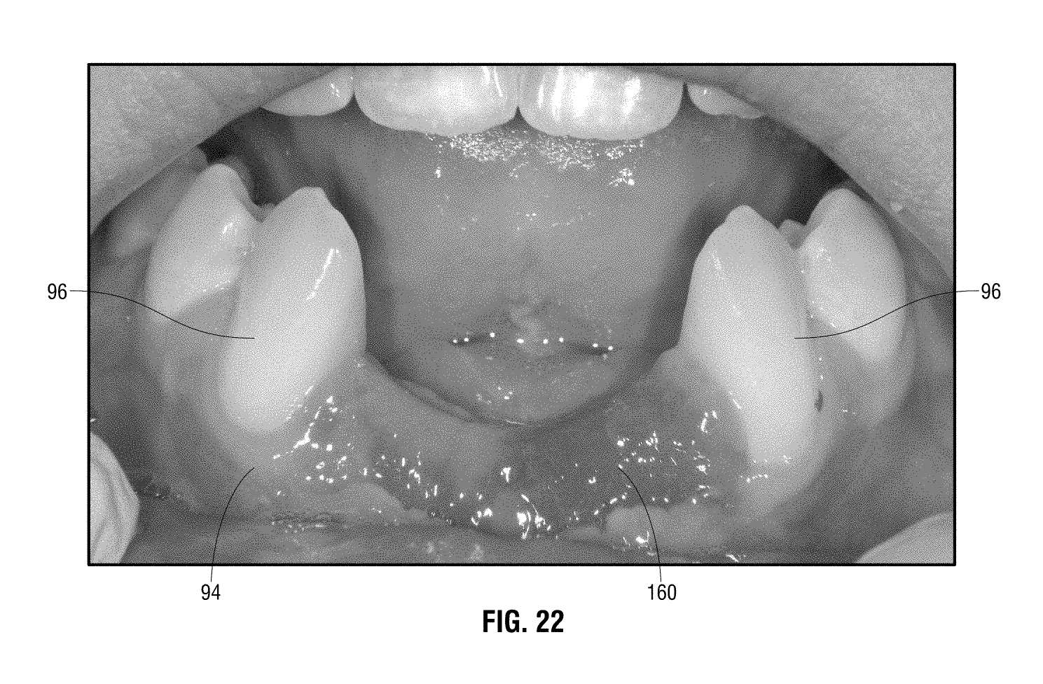

After the bone regeneration stage, the denture may be removed to show the growth of the gingival tissue 160 over occlusive barrier 120, as per FIG. 22. Following design and fabrication, the dentist/physician receives the occlusive barrier. This step includes inspecting barrier marking to ensure matches individualized patient implant order marking, as well as confirming the fit of the barrier against the jaw model. If the occlusive barrier is acceptable, it is fitted to the jaw model and the practitioner schedules the implant procedure. During this stage, also a 30-gauge endodontic cannula should be used to test patency of the irrigation canal by flowing water into the canal.

Prior to implant procedure, steam sterilization per facility protocols. Barrier must be steam sterilized prior to implant per facility protocols. Prior to implant procedure consideration of patient condition to ensure still acceptable for Barrier implant. Be sure that the patient's dentition and jaw has not changed since the CT was taken. New restorations that will affect the seating of the barrier. Drifting or tooth movement. Periodontal disease. Unanticipated procedures (bone grafting, osseous resection, orthognathic surgery, traumatic event).

Here described is the procedure for implanting occlusive barrier 10. The patient's intra and extra oral environment should be disinfected with chlorhexidine or some antiseptic. A full-thickness flap is exposed in the augmentation area. Wide access is needed for complete access 3-4 mm beyond the margin of the occlusive barrier. This may include reflecting 3 teeth beyond the augmentation area and/or placing vertical releasing incisions. Any residual soft tissue should be removed via curettes, back-action hoes, or a piezoelectric device. Cortical perforation to expose the trabecular bone is not recommended.

After opening of the sterile pouch under continuous sterile conditions, the barrier fit is verified. Be sure there is no or minimal movement of the barrier when seated. The blood clot is place on the ridge and against the intaglio surface of the barrier and re-seated.

Fixation screws are placed according to manufacture directions. The irrigation canal should be sealed with Teflon plug. The flap is approximated, starting from the most distal and mesial edges of the flap. No periosteal releasing incisions are needed because complete flap closure is not necessary. Non-resorbable or slowly-resorbing suture is recommended with minimal bacterial wicking-suture material such as e-PTFE, Polypropylene, or poliglecaprone 25.

Post-implantation of occlusive barrier 10 follow-up on patient. Post-operative visits should be scheduled at day 3, week 1, week 3, week 6, week 8, week 12, and week 16. The site should be irrigated with chlorhexidine or 30% hydrogen peroxide around the flap margins beginning on day 3 but not in the irrigation canal until week 6. The irrigation canal should be irrigated into on weeks 6, 8, 12, and 16.

Determination of when healing sufficient to remove Barrier. Depending on the patient's level of hygiene and compliance, the barrier should remain for a minimum of 4 months. If the patient experiences pain with putrid suppuration (plasmatic flow of clear liquid is normal). Antibiotic combination of Amoxicillin (or Clindamycin) with Metronidazole should be used. More frequent post-operative visits may be indicated with Chlorhexidine/Hydrogen peroxide into the canal.

Early removal of the barrier may be indicated. Removal of the barrier can usually be accomplished with infiltration of local anesthesia. Remove the fixation screws being sure to all the screws are accounted for. The barrier can be removed with a periosteal elevator or curette by gently lifting each edge. Debris and materia alba underneath the barrier is normal and can be easily rinsed away. If additional augmentation is desired, the barrier can be re-sterilized and replaced with another clot and new, larger screws.

The residual buccal and lingual flap can be contoured by denuding the inner flap and sutured together over the ridge. The barrier should be disposed of in biomaterial waste. Four to six months is observed to allow for soft and hard tissue maturation. The unkeratinized tissue on the new ridge will keratinized during this time. Laser contouring of the residual flap can be done at the time of implant placement, if indicated. The implant(s) should be placed via flapless technique as to avoid interruption of endosteal bone maturation.

The method of the present disclosure is characterized by requiring a single surgery at the receiver site of occlusive barrier 10, with no need for bone filler or graft of any type. Because it does not require any fillings, the osteoconductive capacity of titanium allows the blood vessels to construct scaffolding for the osteogenic cells in the clot, giving the proper conditions for the growth of the new bone.

Likewise, in the absence of any filler, biological mechanisms do not require foreign body resorption. Therefore, the new bone formation is commenced once the occlusive barrier is placed, i.e., the time needed for tissue regeneration is much shorter.

By the technology used in the design and manufacturing process of the biomedical device, it makes it possible to understand the anatomy of the surgical field in its three dimensions prior to the surgery, even making possible the operation in a virtual way.

As described, the present disclosure provides the use of digitalized design and manufacturing processes (CAD-CAM), the software required for printing the occlusive barrier customized for the patient. Since the occlusive barrier is a custom-manufactured device, the adaptation and the peripheral seal will completely prevent the entry of soft tissue and bacteria, a situation that makes it possible to guarantee the success of the treatment.

The present disclosure is characterized by the possibility of placing the implants at the same surgical moment as the occlusive barrier, so that new bone tissue is formed at the same time as the osseointegration thereof is carried out with the implants, leading to a very significant gain in time for the initiation of patient rehabilitation.

Occlusive barrier 10 provides a temporarily, non-resorbable, implantable material for use as a space-making barrier in the treatment and augmentation of alveolar ridge in accordance with guided tissue regeneration principle. The BBS is supplied non-sterile and must be steam sterilized per facility protocol prior to implantation. The BBS may only be installed by Presently disclosed-trained personnel.

Occlusive barrier 10 is part of the concept of guided tissue regeneration and is related to the exclusion of tissues, called compartmentalization. Quicker growing soft tissues eliminate any chance of hard tissue growth. By completely excluding soft tissues from the site of augmentation, bone will be allowed to regenerate in a protected environment. Additionally, the custom-fabricated barrier is rigid for stabilized bone growth.

Extensive research has shown that bone graft materials underneath the occlusive barrier may impede natural bone growth. However, the addition of extracellular matrix has shown benefit as a bio scaffold. Depending on the severity of bone loss, early woven bone will form in four to six months. Once the barrier is removed, the formed bone will mature for implant placement in four to six months.

The method and system of the present disclosure provide for blood collection such as with vacutainers with clot activators (Gold, Red/Black, Red plastic, Orange or Grey/Yellow). To aid timely clot formation, the tube can be heated either with a warm bath (40° C.) or body temperature. Per the design envelope noted above, are there any type of patients who would be excluded for user dentist/physician. Exclusion criteria is based on the dentist/physician's clinical judgment, as would normally preclude the patient from surgical therapy (i.e., non-compliance, poor oral hygiene, medically compromised) patients that would be precluded from ridge augmentation surgery, uncut to make the barrier difficult to seat, mobile teeth, existing infection or periodontal medical problems that would contraindicate surgical therapy, poor home care and non-compliance, case selection that would normally contraindicate surgery.

Minimal size is likely to treat a single tooth, but may need to be at least the size of three teeth (i.e., eight mm). The maximum size would be used for a full arch (maxillary) ridge around 40 mm. Accessories include sterile techniques and universal precautions must be observed as with any invasive surgical procedure. No additional armamentarium is needed beyond instruments used for guided bone regeneration and fixation. Implant screws include any fixation screw system can be used. The specifications for diameter must be given by the ordering dentist/physician so that the appropriately rendered access hole can be incorporated into the CAD. The recommended diameter of the screw should be 1.5-2.75 mm. The length of the screw is based on the surgeon's clinical judgement, as to avoid critical structures.

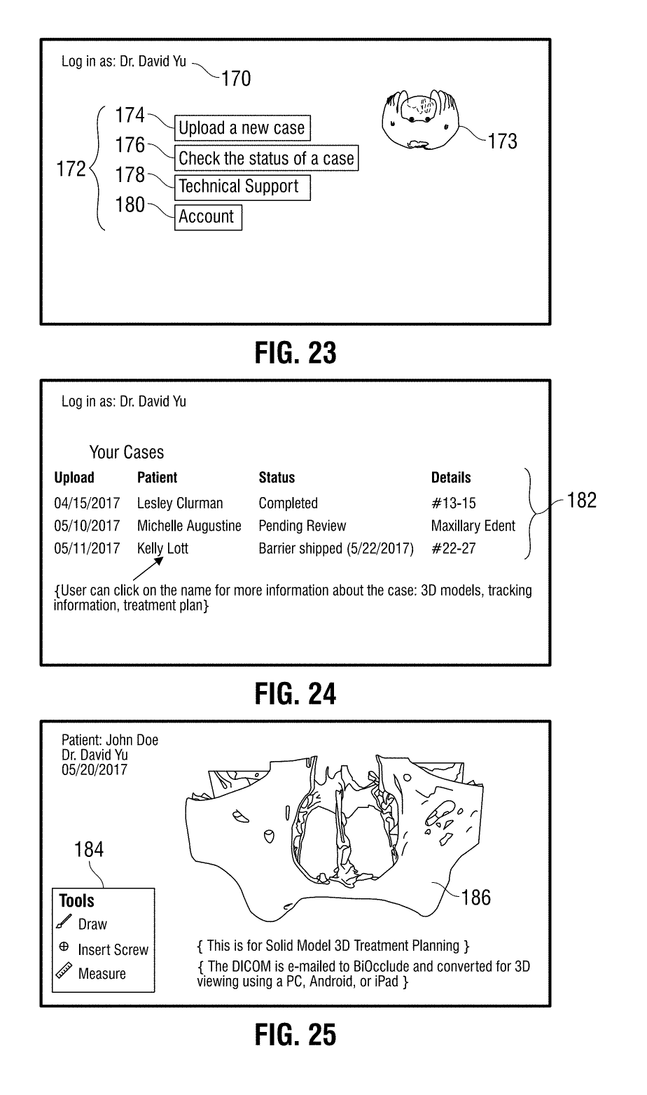

FIGS. 23 through 33 illustrate use of the occlusive barrier design and management software for the process of the present disclosure. Referring to FIG. 23 appears a computer screen 170 providing an occlusive Barry your design and management process. Referring to occlusive barrier and jaw configuration 174, the user may upload a new case at function 174, check the status of an existing case at function 176, achieve technical support as necessary, and manage his own account for occlusive barrier process 40. Referring to FIG. 24, the doctor's cases, available upon selection of the “Check the Status of a Case” function 174, shows the various patients and cases being attended to by the practicing physician. The information includes the upload date of the cases into the software, the patient's identification or name, the condition or status of the case, and any relevant detail details that the practicing doctor may find beneficial in service to the patient.

FIG. 25 illustrates how the software provides the ability to manage a new case. The screen for FIG. 25 appears by selection of “Upload a New Case” function 174. The new case screen includes the tool section 184 and a 3-D CT scan configuration 186. The displayed CT scan image 186 is the basis for communication between the barrier center, the barrier manufacturer, the practicing the physician and the patient.

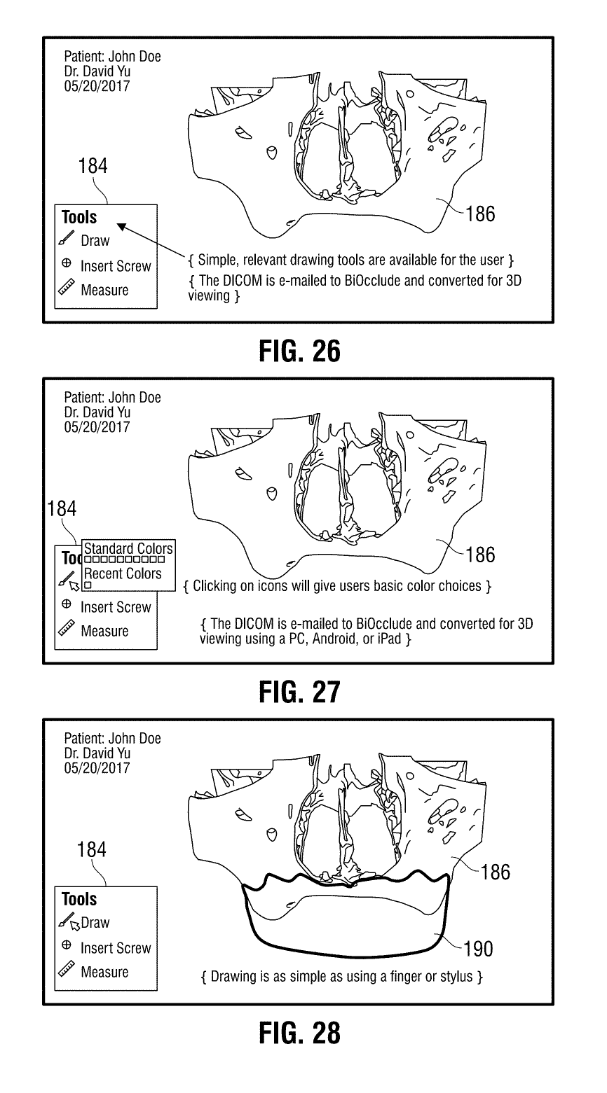

FIGS. 26 through 33 illustrate the features of the case design function other presently disclose process. So, referring to FIG. 24 CT scan 186 appears and maybe controlled or used using tools 184. These tools include a draw function and insert screw function for the purpose of designating the position of a set screw for an occlusive barrier 10, as well as a measuring tool for determining distances between respective components within the plan. Referring to FIG. 27, CT scan 186 may be modified or controlled using the drawing function from tool section 184. Thus, upon the doctor using the draw function, as FIG. 28 shows, he may draw an occlusive barrier at the bottom of the jaw CT scan 186. This appears as diagram 190 at the bottom of jaw CT scan 186.

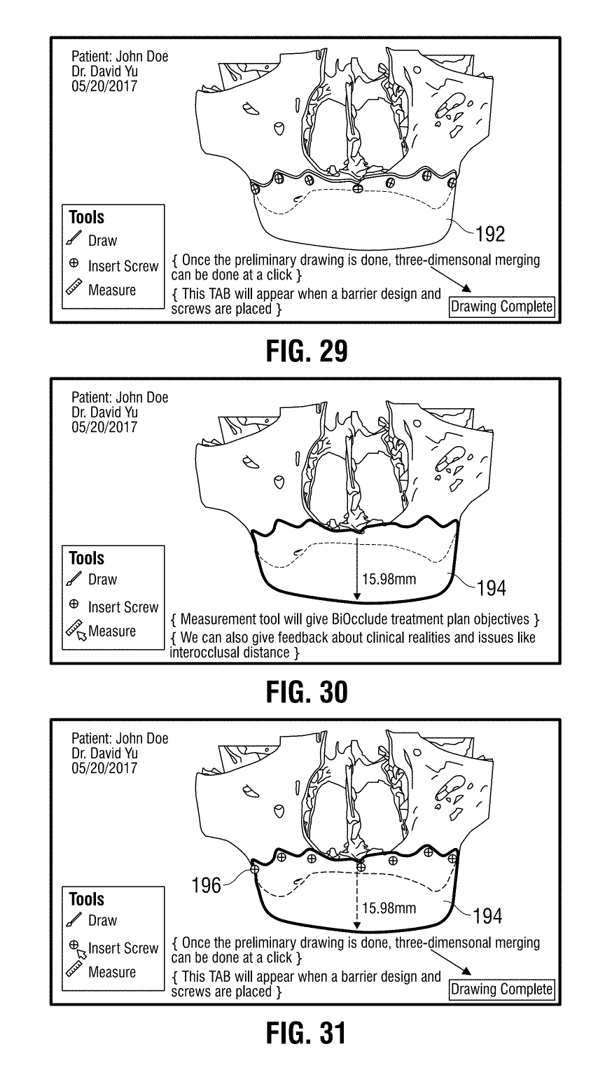

FIG. 29 shows how the CT scan software of the present disclosure takes the sketch from the doctor and converts the doctor's drawing into a three-dimensional template for the patient according to the physical parameters of the jaw CT scan 186. Thus, the occlusive barrier template 192 may be formed. This will be the beginning of the occlusive barrier 10 formation process.

FIG. 30 illustrates how the measurement tool from tool box 184 may be applied to the design to improve for a very the drawing by the doctor to create a template consistent with a treatment plan needed for the particular patient. In addition, the occlusive barrier software can provide feedback about clinical problems that are important for the barrier formation and bone regeneration process.

FIG. 31 further provides the next step following the formation of a preliminary drawing for occlusive barrier 10 once the preliminary drawing is done. Then, three-dimensional merging between occlusive barrier 10 design and the jaw CT scan can occur. Once the barrier design is complete, the barrier software can form the three-dimensional barrier template. Then, the software will indicate to the user that the drawing is complete.

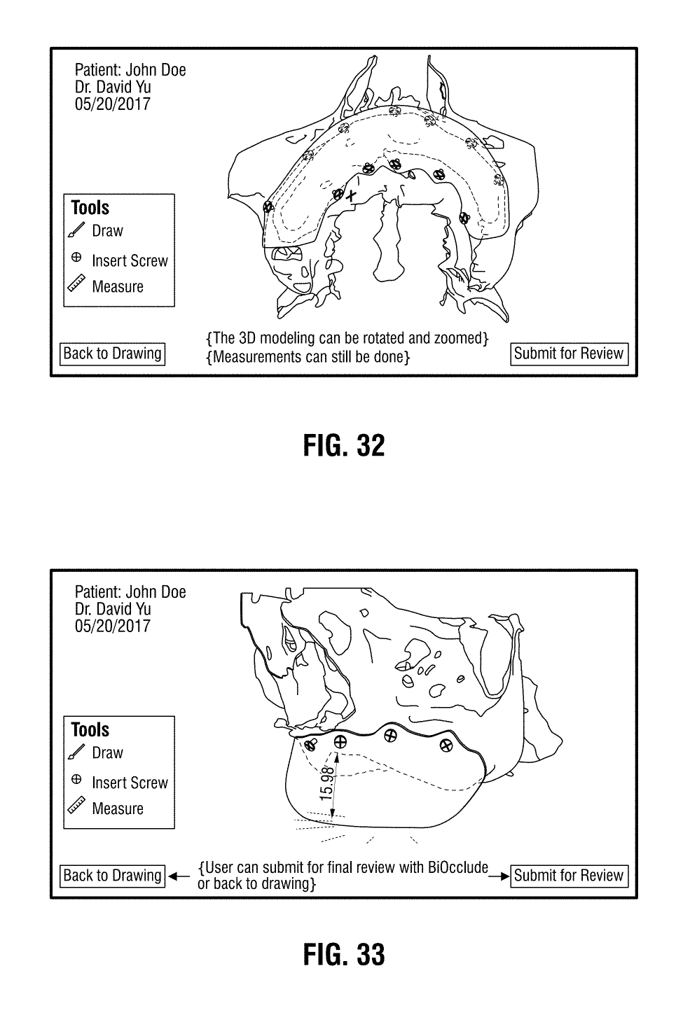

With the drawing complete, the occlusive barrier formation software allows three-dimensional modeling in rotation of the jaw CT scan and occlusive barrier configuration so that all participating doctor designer and manufacturer can see that the design will work as needed. The three-dimensional viewing is seen in FIGS. 32 and 33. Once all concerned are satisfied that the design will work, the user can submit the design for final review to the barrier center for approval and formation of our manufacturing of the inclusive barrier.

Here, the user can click on the name for more information about the case: 3D models, tracking information, treatment plan. This is for Solid Model 3D Treatment Planning. The DICOM is e-mailed to presently disclosed and converted for 3D viewing using a PC, Android, or iPad. Simple, relevant drawing tools are available for the user. The DICOM is e-mailed to the occlusive barrier designer and converted for three-dimensional viewing. Clicking on icons will give users basic color choices. Drawing is as simple as using a finger or stylus.

The three-dimensional modeling can be rotated and zoomed. Measurements can still be done. Submit for Review. User can submit for final review with presently disclosed or back to drawing. Submit for Review. Various tabs will appear when barrier design and screws are placed. Measurement tool will give presently disclosed treatment plan objectives. We can also give feedback about clinical realities and issues like interocclusal distance. Once the preliminary drawing is done, three-dimensional merging can be done at a click. At this point the drawing is complete.

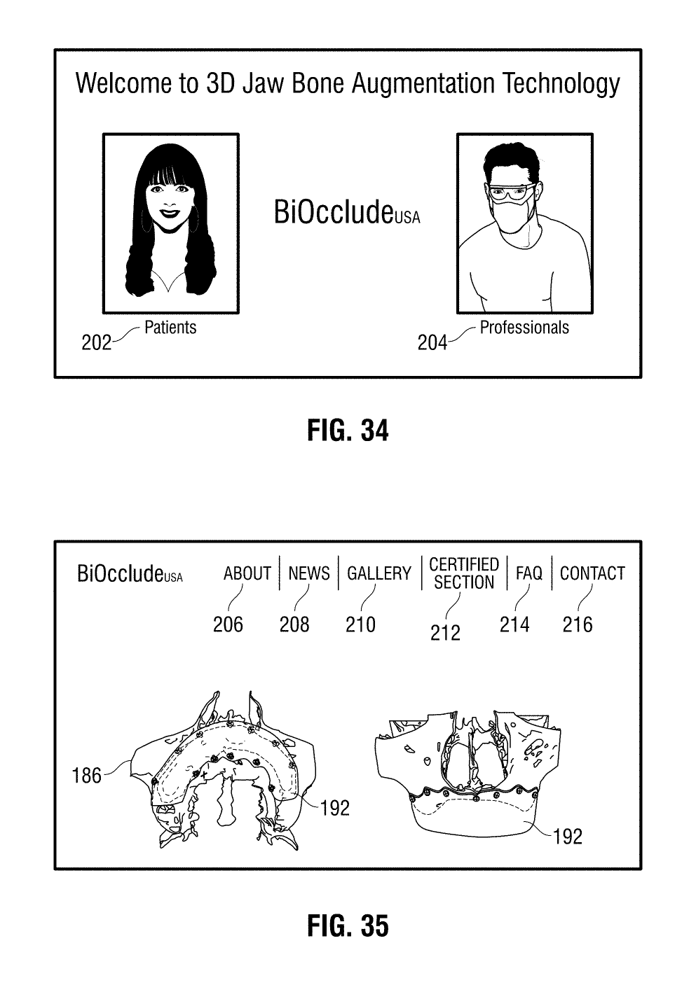

In addition to the occlusive barrier process 40 software for the design of the occlusive barrier, the present method and system further include a web-based interface whereby patients and professionals can communicate with the plan and design for conducting the surgery and placement of the occlusive barrier 10 for bone regeneration. Thus, FIGS. 34 through 37 illustrate the functionality of the web-based interface. Referring to FIG. 34, the website homepage includes access to patients at 202 and professionals at 204. Through both website access points 202 and 204 may appear the three-dimensional images of the CT scan and occlusive barrier. This appears in FIG. 35. For certified professionals, the barrier center software website provides information on how to order or access the services of the barrier center in the formation of occlusive barrier 10. This interface receives email and allows for a password whereby the functionality of the software in general can be managed.

At FIG. 37, the barrier center software allows for the uploading of CT scan files to support use of the design process. This information includes the name of the doctor the patient name and any patient ID, such as data birth. The digital files necessary for the use of the disclosed process may be added through this interface of FIG. 37. FIG. 37 further details requirements of the digital files. Including their digital requirements as well as scanning parameters necessary for use of the process.

Because of the highly-specialized technology, only surgeons with extensive experience and training will be able to order presently disclosed barriers. Interested surgeons must apply and be approved to take the certification course. After mentored cases are completed with documentation, the surgeon will be certified to order the barriers. Please visit our certified section for more information. Surgeons must apply and be approved to take the certification course. After review, course information will be sent to you by e-mail. Final certification will allow the surgeon to order presently disclosed barriers using a personalized protected portal which is HIPPA-compliant.

Patients that have severe atrophy of the jaw bones who are not candidates for traditional rehabilitation with implants now have hope. Our doctors and engineers custom design each case for the certified surgeon's approval.

The present disclosure further includes a software application that is fully digital and seamless. This application is easy for surgeons to treatment plan and order our product. The application takes advantage of various familiar platforms (iOS, Android, Google, etc.). Payment is easy because credit card authorization will be linked to each account. It will also allow for pre-paid bulk purchases. The customer can track the progress of each case.

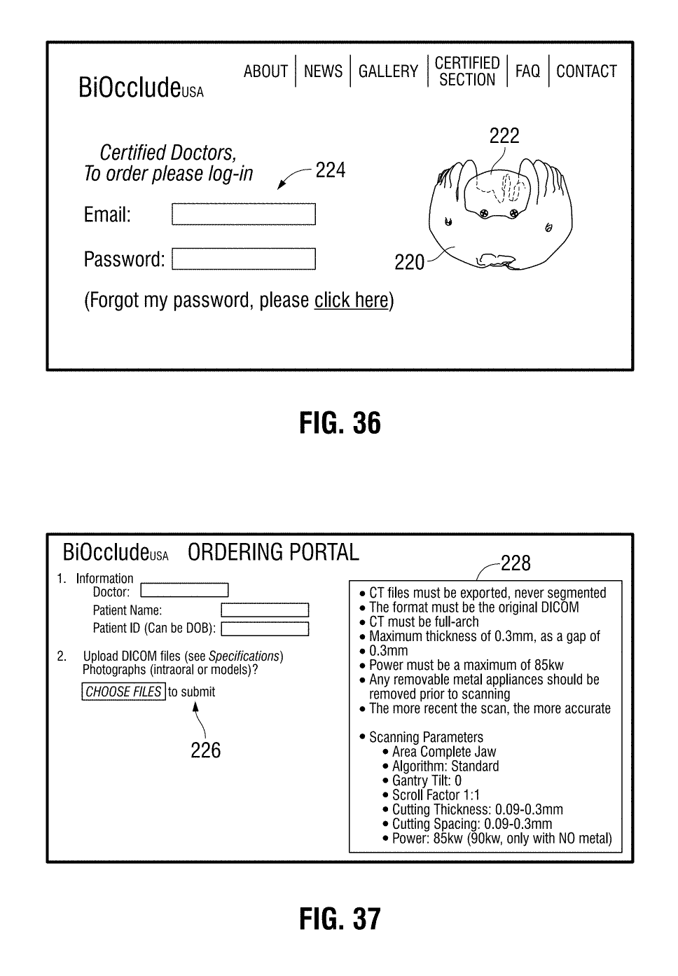

In particular, FIG. 34 illustrates that the website interface is available to patients as well as professionals. FIG. 35 provides the same types of three-dimensional image construction as appearing in the software for managing and constructing the occlusive barrier. FIG. 36 provides an aspect of the website exclusively for certified doctors are professionals in designing and receiving and managing the results of the occlusive beer or your formation process of the present disclosure. FIG. 37 provides an exemplary ordering portal for use with the subject matter of the present disclosure whereby CT scan files can be uploaded and made part of the ordering process for using the design construction automation and visualization aspects of the present we disclosed method and system.

In summary, the present disclosure provides a method, system, and integrated medical system for bone tissue regeneration in association with a predetermined dental bone structure, comprising the steps of obtaining a computerized tomography scan of a dental bone structure on which to regenerate bone tissue. A three-dimensional model formed from the computerized tomography scan digitally represents the dental bone structure. The method and system present the three-dimensional model on a computer display. A treatment plan corresponds to the three-dimensional model. The method and system receive a design order relating to the treatment plan for forming an occlusive barrier for covering the portion of the dental bone structure whereupon to regenerate bone tissue. An occlusive barrier forms from a biocompatible material in accordance with said design order. An osteoconductive material is placed within an interior volume of the occlusive barrier for associating with and from which may form flesh and regenerated bone tissue via osteoconduction in association with the portion of the dental bone structure covered by said occlusive barrier. The method and system further include fixing the occlusive barrier and the osteoconductive material to the dental bone structure for a time period sufficient for regeneration of bone tissue associated with dental bone structure. The occlusive barrier is removed from the dental bone structure upon said bone tissue regeneration reaching a predetermined stage.

The disclosed subject matter further includes an occlusive barrier for osteoconductive bone tissue regeneration in association with a predetermined dental bone structure. The occlusive barrier formed by performing the steps of obtaining a computerized tomography scan of a dental bone structure on which to regenerate bone tissue. A three-dimensional model from said computerized tomography scan for digitally representing the dental bone structure. The occlusive barrier may include a plurality of irrigation channels for permitting flushing of said interior volume during regeneration of bone tissue associated with dental bone structure.

The occlusive barrier may be specified using a computer aided design application and manufactured by a titanium laser sintering process. The occlusive barrier further includes a space between the predetermined dental bone structure tissue and gingival tissue for promoting bone growth from a layer of stem cells covering an outer endosteum outer surface of the dental bone structure. The occlusive barrier may be formed from a plurality of pieces printed for associating into said occlusive barrier covering said portion of the dental bone structure whereupon to regenerate bone tissue. The occlusive barrier is subjected to a heat treatment for alleviating molecular stress and increasing occlusive barrier ductility and strength and may be subjected to a surface sandblasting treatment for forming a surface porosity promoting osteoconduction and blood vessel formation. Furthermore, the occlusive barrier may be subjected to an anodizing treatment for cleaning organic and inorganic residues from surfaces of said occlusive barrier.

The detailed description set forth herein in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments.

This detailed description of illustrative embodiments includes specific details for providing a thorough understanding of the presently disclosed subject matter. However, it will be apparent to those skilled in the art that the presently disclosed subject matter may be practiced without these specific details. In some instances, well-known structures and devices are shown in block diagram form in order to avoid obscuring the concepts of the presently disclosed method and system.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein, but is to be accorded the widest scope consistent with the principles and novel features disclosed herein. It is contemplated that additional embodiments are within the spirit and true scope of the disclosed subject matter.

What is claimed is:

1. A method for bone tissue regeneration in association with a predetermined dental bone structure, comprising the steps of:
obtaining a computerized tomography scan of a dental bone structure on which to regenerate bone tissue;
forming a three-dimensional model from said computerized tomography scan for digitally representing the dental bone structure;
presenting said three-dimensional model on a computer display;
receiving a treatment plan corresponding to said three-dimensional model;
receiving a design order relating to said treatment plan for forming an occlusive barrier for covering the portion of the dental bone structure whereupon to regenerate bone tissue;
forming an occlusive barrier from a biocompatible material in accordance with said design order;
placing an osteoconductive material within an interior volume of said occlusive barrier for associating with and from which may form flesh and regenerated bone tissue via osteoconduction in association with the portion of the dental bone structure covered by said occlusive barrier;
fixing said occlusive barrier and said osteoconductive material to the dental bone structure for a time period sufficient for regeneration of bone tissue associated with dental bone structure, and
removing said occlusive barrier from said dental bone structure upon said bone tissue regeneration reaching a predetermined stage.

2. The method of claim 1, further comprising the step of obtaining a computerized tomography scan of a dental bone structure on which to regenerate bone tissue through an internet web application.

3. The method of claim 1, further comprising the step of forming a three-dimensional model from said computerized tomography scan for digitally representing the dental bone structure using a three-dimensional printing software application.

4. The method of claim 1, further comprising the step of presenting said three-dimensional model on a computer display for viewing said three-dimensional model from a multitude of three-dimensional perspectives.

5. The method of claim 1, further comprising the step of receiving a treatment plan corresponding to said three-dimensional model from a remote location corresponding to a certified dental surgeon office at a remote location.

6. The method of claim 1, further comprising the step of receiving a design order relating to said treatment plan for forming an occlusive barrier for covering the portion of the dental bone structure whereupon to regenerate bone tissue, said design order specifically relating to an individual patient for whom said occlusive barrier.

7. The method of claim 1, further comprising the step of forming an occlusive barrier from a biocompatible material in accordance with said design order;

8. The method of claim 1, further comprising the step of placing a blood clot as said osteoconductive material within an interior volume of said occlusive barrier for associating with and from which may form flesh and regenerated bone tissue via osteoconduction in association with the portion of the dental bone structure covered by said occlusive barrier.

9. The method of claim 1, further comprising the step of applying set screws through said occlusive barrier and into said dental bone structure for fixing said occlusive barrier and said osteoconductive material to the dental bone structure.

10. A system for bone tissue regeneration in association with a predetermined dental bone structure, comprising the steps of:
a computerized tomography scan of a dental bone structure on which to regenerate bone tissue;
a three-dimensional model formed from said computerized tomography scan for digitally representing the dental bone structure;
a computer display for presenting said three-dimensional model;
a treatment plan corresponding to said three-dimensional model;
a design order relating to said treatment plan for forming an occlusive barrier for covering the portion of the dental bone structure whereupon to regenerate bone tissue;
an occlusive barrier formed from a biocompatible material in accordance with said design order;
an osteoconductive material for placing within an interior volume of said occlusive barrier for associating with and from which may form flesh and regenerated bone tissue via osteoconduction in association with the portion of the dental bone structure covered by said occlusive barrier;
said occlusive barrier and said osteoconductive material fixed to the dental bone structure for a time period sufficient for regeneration of bone tissue associated with dental bone structure, and
said occlusive barrier further removed from said dental bone structure upon said bone tissue regeneration reaching a predetermined stage.

11. The system of claim 10, further comprising the step of obtaining a computerized tomography scan of a dental bone structure on which to regenerate bone tissue through an internet web application.

12. The system of claim 10, further comprising the step of forming a three-dimensional model from said computerized tomography scan for digitally representing the dental bone structure using a three-dimensional printing software application.

13. The system of claim 10, further comprising the step of presenting said three-dimensional model on a computer display for viewing said three-dimensional model from a multitude of three-dimensional perspectives.

14. The system of claim 10, further comprising the step of receiving a treatment plan corresponding to said three-dimensional model from a remote location corresponding to a certified dental surgeon office at a remote location.

15. The system of claim 10, further comprising the step of receiving a design order relating to said treatment plan for forming an occlusive barrier for covering the portion of the dental bone structure whereupon to regenerate bone tissue, said design order specifically relating to an individual patient for whom said occlusive barrier.

16. The system of claim 10, further comprising the step of forming an occlusive barrier from a biocompatible material in accordance with said design order;

17. The system of claim 10, further comprising the step of placing a blood clot as said osteoconductive material within an interior volume of said occlusive barrier for associating with and from which may form flesh and regenerated bone tissue via osteoconduction in association with the portion of the dental bone structure covered by said occlusive barrier.

18. The system of claim 10, further comprising the step of applying set screws through said occlusive barrier and into said dental bone structure for fixing said occlusive barrier and said osteoconductive material to the dental bone structure.

19. An occlusive barrier for osteoconductive bone tissue regeneration in association with a predetermined dental bone structure, said occlusive barrier formed by performing the steps of:
obtaining a computerized tomography scan of a dental bone structure on which to regenerate bone tissue;
forming a three-dimensional model from said computerized tomography scan for digitally representing the dental bone structure;
presenting said three-dimensional model on a computer display;
receiving a treatment plan corresponding to said three-dimensional model;
receiving a design order relating to said treatment plan for forming said occlusive barrier for covering the portion of the dental bone structure whereupon to regenerate bone tissue;
forming said occlusive barrier from a biocompatible material in accordance with said design order;
placing an osteoconductive material within an interior volume of said occlusive barrier for associating with and from which may form flesh and regenerated bone tissue via osteoconduction in association with the portion of the dental bone structure covered by said occlusive barrier;
fixing said occlusive barrier and said osteoconductive material to the dental bone structure for a time period sufficient for regeneration of bone tissue associated with dental bone structure, and
removing said occlusive barrier from said dental bone structure upon said bone tissue regeneration reaching a predetermined stage.

20. The occlusive barrier of claim 19, further comprising a plurality of irrigation channels for permitting flushing of said interior volume during regeneration of bone tissue associated with dental bone structure.

21. The occlusive barrier of claim 19, further specified using a computer aided design application and manufactured by a titanium laser sintering process.

22. The occlusive barrier of claim 19, further comprising a space between the predetermined dental bone structure tissue and gingival tissue for promoting bone growth from a layer of stem cells covering an outer endosteum outer surface of the dental bone structure.

23. The occlusive barrier of claim 19, wherein said occlusive barrier further comprises a plurality of pieces printed for associating into said occlusive barrier covering said portion of the dental bone structure whereupon to regenerate bone tissue.

24. The occlusive barrier of claim 19, wherein said occlusive barrier is subjected to a heat treatment for alleviating molecular stress and increasing occlusive barrier ductility and strength.

25. The occlusive barrier of claim 19, wherein said occlusive barrier is subjected to a surface sandblasting treatment for forming a surface porosity promoting osteoconduction and blood vessel formation.

26. The occlusive barrier of claim 19, wherein said occlusive barrier is subjected to an anodizing treatment for cleaning organic and inorganic residues from surfaces of said occlusive barrier.

27. The occlusive barrier of claim 19, wherein said occlusive barrier further comprises a thickness of between 0.3 and 0.6 millimeters.
